(19) United States
(12) Patent Application Publication
(10) Pub. No.: US 2006/0153733 A1
(43) Pub. Date: Jul. 13, 2006

(54) Door handle sanitizer system and apparatus

(76) Inventor: Simon Sassoon, New York, NY (US)

Correspondence Address: DARBY & DARBY P.C., P. O. BOX 5257, NEW YORK, NY, 10150-5257, US

(21) Appl. No.: 11/104,292

(22) Filed: Apr. 11, 2005

Related U.S. Application Data
(60) Provisional application No. 60/642,792, filed on Jan. 10, 2005.

(51) Int. Cl. A61L 2/18

(52) U.S. Cl. 422/28; 422/292

(57) ABSTRACT

An apparatus and method for sanitizing door handles includes dispensing a germicide, such as a disinfectant, antibacterial solution or cleansing agent, onto a door handle at controlled intervals. The door handle sanitizer further includes a source of liquid or gaseous disinfectant, a spray nozzle configured to convey liquid or gaseous disinfectant from the source to the nozzle and for dispensing the liquid or gaseous disinfectant onto the handle, and a controller for causing the liquid or gaseous disinfectant to be conveyed to the handle in intervals, wherein the intervals are responsive to an external condition and the controller if free of any manual activation.

### PRIORITY

This application claims the benefit of U.S. Provisional Application Ser. No. 60/642,792, filed Jan. 10, 2005.

### FIELD OF THE INVENTION

The present invention relates to an apparatus and method for rendering germ-free (sanitizing) door handles, and more particularly, to a device for dispensing a germicide, such as a disinfectant, antibacterial solution or cleansing agent, onto a door handle at controlled intervals.

### BACKGROUND OF THE INVENTION

Many people are reluctant to use public restrooms due to actual or perceived sanitary conditions of those restrooms. However, in some instances, such use is unavoidable.

These people try to avoid touching any surface after they have washed their hands. However, touching a surface of the restroom is nearly unavoidable because sometimes the person must touch the handle of the door to exit the restroom. Some people carry gloves, wipes or the like to use in such public restrooms. Some people take an extra paper towel to use to cover the door handle. All of these techniques work, but are burdensome and not efficient.

Some prior art methods suggest covering a handle of a door so a person opening the door can avoid direct contact with the handle. Covering a door handle, while effective in preventing a person from contacting the door handle during operation of the door, does little to clean or disinfect the door handle. Furthermore, the cover then becomes a source of contamination, germs, bacteria and the like. Therefore, there is a need for efficiently and effectively sanitizing the handle of a door. This typically requires a person to carry a liquid spray bottle into a room, spray a disinfectant or antibacterial liquid onto the door handle and then wipe the handle clean. This procedure can be cumbersome and inefficient, requiring a person to carry items with him or her for the cleaning procedure.

Still further, some people, often nicknamed germephobes, wonder when the last time a door handle was cleaned, and even if there is some form of protection for this person, they are uncomfortable touching the door handle. These people are not satisfied by the mere existence of some means for cleaning or sanitizing the door handle, rather, they might prefer to know that such a cleansing device is activated at intervals in response to certain conditions or according to a prescribed cleaning schedule, such as every several minutes. Therefore, there is a need for efficiently cleaning and sanitizing a door handle at prescribed time intervals in response to certain conditions, i.e., according to a predetermined cleaning schedule, opening and closing of the door, passing of an individual in the vicinity of the door knob, turning on/off of the lights, etc.

### SUMMARY OF THE INVENTION

In accordance with one aspect of the invention, a door handle sanitizer for disinfecting a door handle includes a source of liquid or gaseous disinfectant, a spray nozzle configured to convey liquid or gaseous disinfectant from the source to the nozzle and for dispensing the liquid or gaseous disinfectant onto the handle, and a controller for causing the liquid or gaseous disinfectant to be conveyed to the handle in intervals, wherein the intervals are responsive to an external condition and the controller if free of any manual activation.

In accordance with another aspect of the invention, the door handle sanitizer includes a mount suitable to connect the sanitizer to the door. The spray nozzle is configured to convey a prescribed amount of the liquid or gaseous disinfectant from the source to the nozzle and dispense the prescribed amount of liquid or gaseous disinfectant onto the handle.

According to yet another aspect of the present invention, the controller includes a timing circuit which activates the spray nozzle to dispense the liquid or gaseous disinfectant at prescribed time intervals on the basis of a predetermined cleaning cycle of the door handle sanitizer.

According to a further aspect of the invention, a method for sanitizing the door handle includes the steps of mounting the door handle sanitizer proximate the door handle and spraying a germicide as an atomized mist from the dispenser onto the door handle in response to an event free of any manual activation.

These and further aspects, features and advantages of the present invention will become more apparent from the following detailed description when taken in connection with the accompanying drawings which show, for purposes of illustration only, a preferred embodiment of the present invention.

### BRIEF DESCRIPTION OF THE DRAWINGS

FIG. 1 is a perspective view a door handle sanitizer according to a first embodiment of the present invention mounted on a door above the door handle;

FIG. 2 is a perspective exploded view of the door handle sanitizer of FIG. 1;

FIG. 3 is an enlarged view of a germicide source and spray actuator of the sanitizer of FIG. 1;

FIG. 4 is a front elevation view of the door handle sanitizer of FIGS. 1-2 absent the outer cover to show interior components;

FIG. 5 is a partial enlarged elevation view of a door handle sanitizer absent the outer cover to show interior components;

FIG. 6 is a partial enlarged elevation view a door handle sanitizer according to a second embodiment of the present invention;

FIG. 7 is a side elevation view of a sanitizer fixed to a portion of a door with its cover opened;

FIG. 8 is a side elevation view of a sanitizer showing removal and replacement of the germicide source; and

FIG. 9 is a perspective view a door handle sanitizer according to a third embodiment of the present invention mounted on a door above the door handle.

### DETAILED DESCRIPTION OF CERTAIN EMBODIMENTS

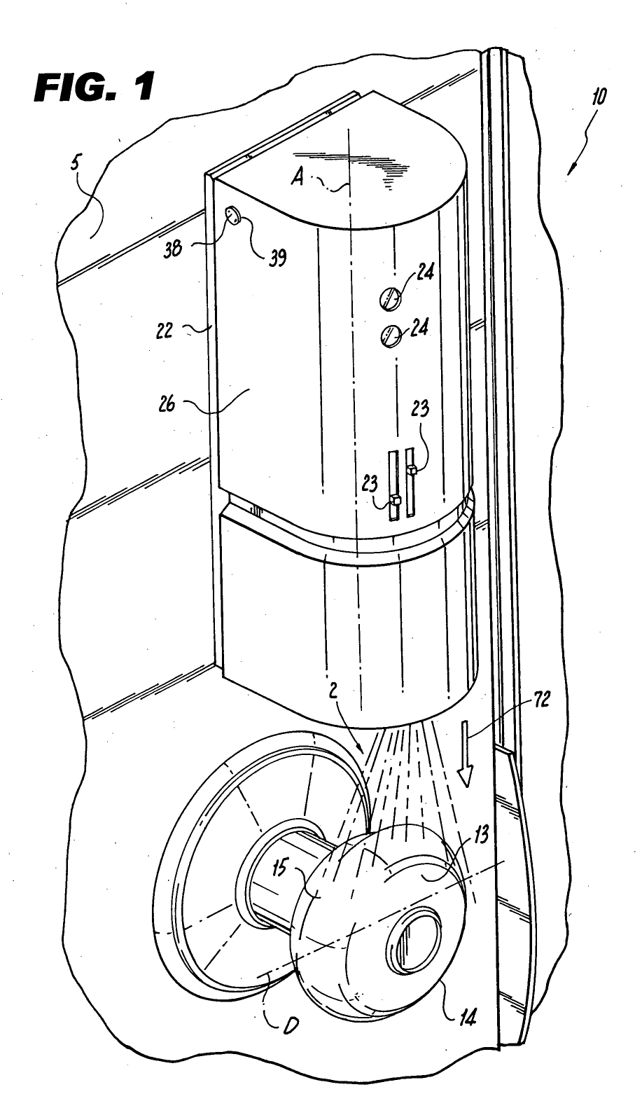

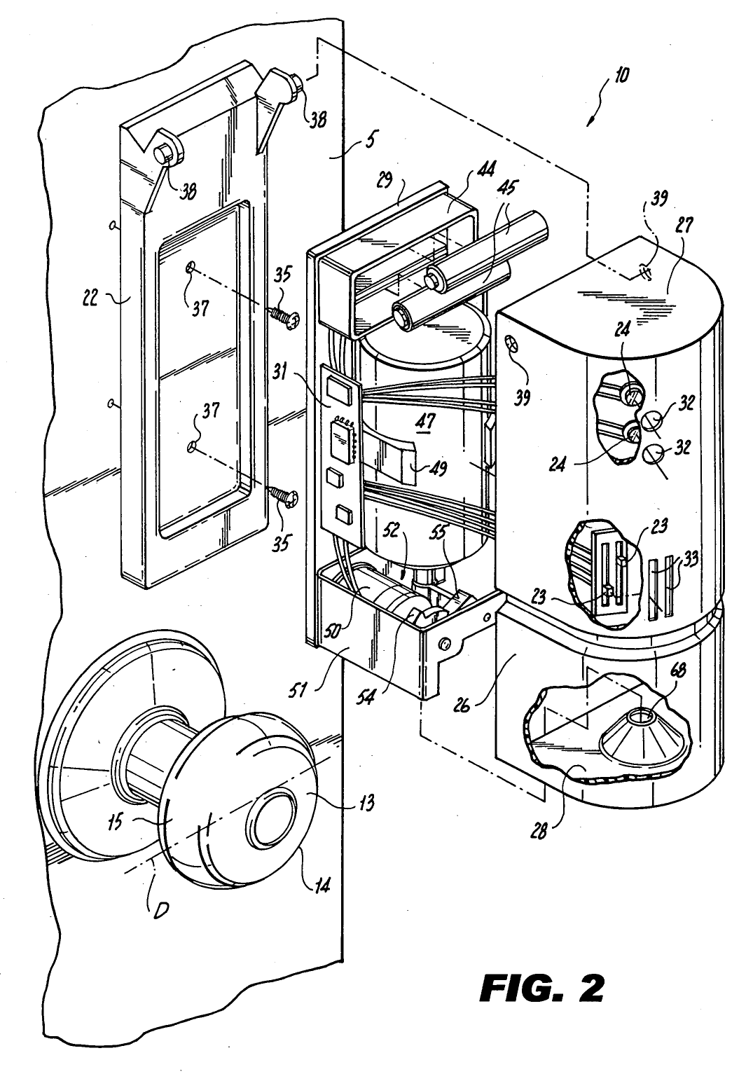

FIG. 1 illustrates a perspective view of a door 5 equipped with a spray dispenser 10 in accordance with a first embodiment of the present invention. Spray dispenser 10 is operable to spray a germicide 2 therefrom to coat, and thereby sanitize, an outwardly extended door handle 14. FIG. 2 illustrates an exploded view of the dispenser 10.

Referring to FIGS. 1 and 2, spray dispenser 10 includes an outer housing 21, base 22, user accessible control switches 23 and visible indicator lights 24 for providing control and status information of dispenser 10.

Spray dispenser 10 is preferably mounted to door 5 above door handle 14 as indicated. Outer housing 21 is hingedly connected to base 22 via prongs 38. Prongs 38 are configured to fit through orifices 39 of housing 21 and allow for rotational movement of the outer housing 21 between a close and opened position. In a closed position, outer housing 21 completely conceals the internal components of dispenser 10, while in an opened position, outer housing 21 provides access to germicide source 47 as well as other internal components contained within dispenser 10, such as batteries 45.

Outer housing 21 of spray dispenser 10 comprises a top wall 27, a bottom wall 28 and, for the purpose of presenting a pleasant appearance, a curved front wall 26. Dispenser 10 also includes a component support wall 29 to which the majority of the internal components of spray dispenser 10 are attached.

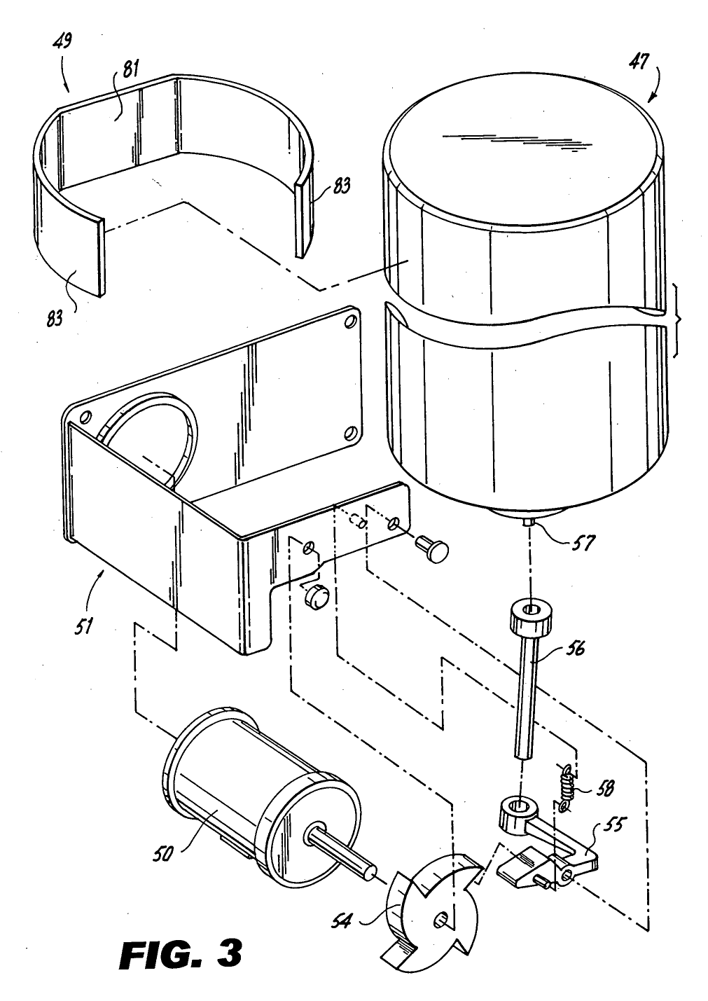

A germicide source 47, preferably in the form of an aerosol can, is removably mounted within spray dispenser 10. Specifically, with reference to FIG. 3, germicide source 47 is retained by a mounting bracket 49 which functions to secure germicide source 47 in place and align its nozzle (valve 57) with elongated nozzle 56 of spray actuator 52. Mounting bracket 49 also provides an efficient means for removal of germicide source 47 in the event replacement is necessary. As illustrated, mounting bracket 49 is preferably U-shaped having a base portion 81 and two arms 83 extending therefrom for securing germicide source 47 therebetween. Base portion 81 is fixed to component support wall 29. Mounting bracket 49 should have flexible properties such that arms 83 are capable of flexing widthwise to allow insertion of germicide source 47 within the confounds of the arms, while also maintaining their shape so as to completely support the weight of germicide source 47. Accordingly, germicide source 47 can “snap in” and “out” of bracket 49 with minimal force and without causing damage to either the bracket of the germicide source and, as stated above, should be sufficient to secure germicide source 47 in place within spray dispenser 10 without the need for additional securing mechanisms. One of ordinary skill in the art would realize that various alternative mounting devices can be employed for securing germicide source within spray dispenser 10, i.e., Velcro straps, ties, belts, clamps, etc., and a spray dispenser 10 according to present invention can be readily adapted to function with any such alternative securing device.

A motor 50, gear 54, arm 55, elongated nozzle 56 and spring 57, herein collectively referred to as the spray actuator 52, are supported within a motor mount bracket 51. Motor mount bracket 51, as with mounting bracket 49 described above, is also fixedly attached to component support wall 29. When outer housing 21 is in a closed position both mounting brackets 51 and 49, germicide source 47 and spray actuator 52 are all shielded from view under the cover of outer housing 21. The relationship between the individual components of spray actuator 52 and their operation in mechanically effecting the release of germicide 2 from source 47 will now be described in detail with reference to FIGS. 3-5.

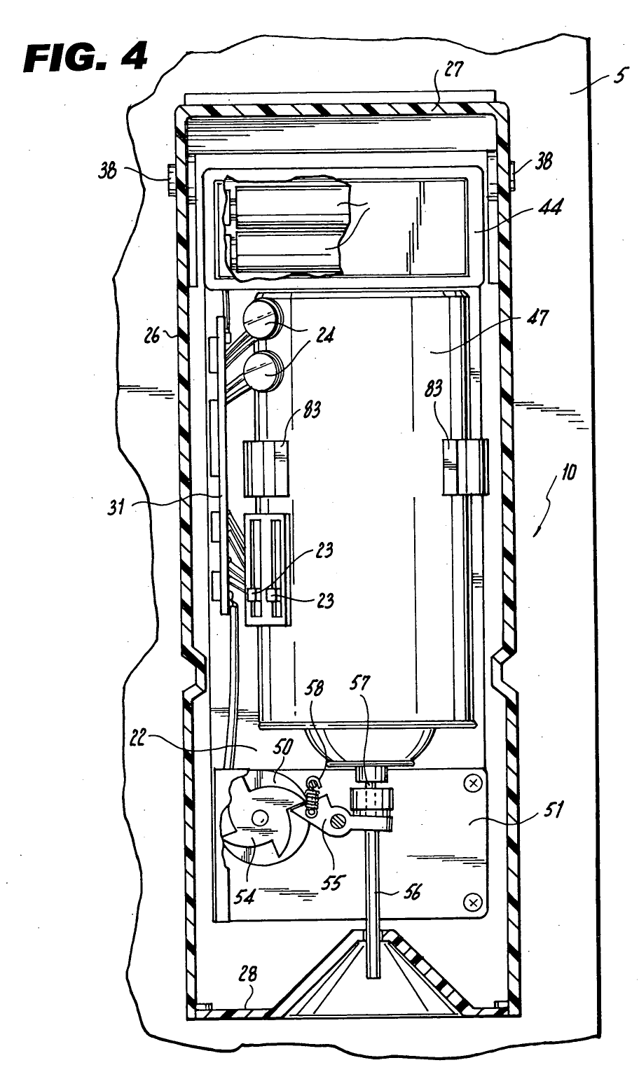

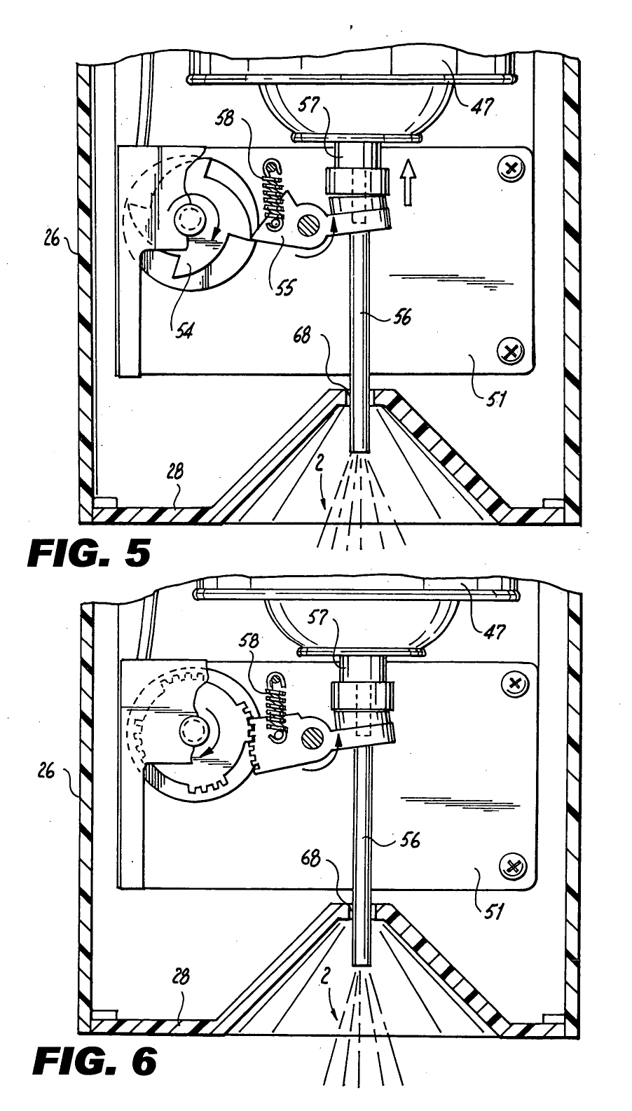

An arm 55 is pivotally mounted to motor mount 51 and supports the elongated nozzle 56. Valve 57 of germicide source 47 is aligned with and fluidly connected to elongated nozzle 56. Referring to FIGS. 3-5, motor 50 is preferably a DC motor and is operable to rotate a gear 54 clockwise through an angle. The clockwise rotation of gear 54 effectuates a counter-clockwise rotational movement in arm 55. Since elongated nozzle 56 is connected to arm 55, the counter-clockwise rotation of the arm 55 forces nozzle 56 upward against valve 57 applying a requisite degree of upward force on valve 57 necessary to temporarily open the valve and release germicide 2 contained in source 47 (FIG. 5). The release of germicide 2 from valve 57 flows into elongated nozzle 56 which directs germicide 2 outward from spray dispenser 10 into the surrounding atmosphere and onto handle 14. A spring 58 is fixedly attached between arm 55 and motor mount bracket 51 and is necessary for returning arm 55 to a normal position (shown in FIG. 4) after making contact with and being forced by gear 54.

It should be understood that motor mount bracket 51 supports the components of spray actuator 52 within spray dispenser 10 without impinging the removal of germicide source 47. As shown in FIG. 4, spray actuator 52 is preferably located beneath germicide source 47 and is configured such that elongated nozzle 56 lines up with and partially encompasses valve 57 when germicide source 47 is in place within spray dispenser 10.

Referring to FIG. 5, gear 54 is preferably configured with equi-distance protrusions or notches along it's peripheral capable of contacting arm 55 and forcing it to pivot as discussed above. Because the forcing of arm 55 by gear 54 results in the opening of valve 57, the longer arm 55 is forced by the gear, the longer valve 57 will remain open and the greater the amount of germicide 2 can be released. The spacing between protrusions on gear 54, the width of each protrusion and the rotational angle through which gear 54 rotates each time motor 50 is activated will all effect how long valve 57 is opened and hence, how much germicidal 2 is released for each given activation. Accordingly, the amount of germicidal released during each activation of spray dispenser 10 can be controlled and an increase or decrease in the amount of germicidal 2 realized by configuring gear 54 so that for each rotation it is contacts and forces arm 55 for a longer or shorter period of time.

In alternative arrangements, the spray activator 52 can be configured with different gears so as to effect a temporary opening of the spray valve 57. Still in further arrangements the spray activator 52 can be configured to pressurize a dose or ampoule of germicide from a reservoir to release the pressurized fluid as a spray onto the handle 14.

Referring to FIG. 2, component support wall 29 defines a battery-containing compartment 44 at a top marginal edge thereof for supporting two AA batteries 45 and includes a control circuit 31 mounted thereto for receiving signals from switches 23, processing the received signals and controlling indicator lights 24 and motor 50. Control circuit 31 is operable to trigger optional indicator lights 24 during the operation of spray dispenser 10, signaling the user of such occurrences as when switches 24 or spray dispenser 10 have been activated. Control circuit 31 is preferably preprogrammed to activate motor 50 at prescribed intervals which are determined by setting switches 23. For instance, switches 23 define a variety of user selectable time intervals and, in response to a specific switch setting, control circuit 31 supplies power to motor 50 at certain intervals. Each time motor 50 is powered it will trigger spray actuator 52 and release germicide 2 from source 47. Since it is known and fairly common that any microcontroller capable of controlling and processing signals indicative of operational time intervals may generally be used, the particular implementation of control circuit 31 has been omitted for clarity. Conventional wires connect control circuit 31 to power (supplied by batteries 45), indicator lights 24, user operable control switches 23 and motor 50.

Referring to FIGS. 4 and 5, elongated nozzle 56 is aligned with a through hole 68 on bottom wall 28 of the housing 21. Through hole 68 functions as an access point through which elongated nozzle 56 exits housing 21. The elongated nozzle 56 is fluidly connected to the germicide source 47 via valve 57 to receive germicide therefrom and is oriented to spray germicide in axial dimension A of housing 21 (FIG. 1) away from the bottom wall 28 of the housing 21 and toward handle 14 of the door 5. This outwardly directed spray 72 should be of a force and breadth to completely cover at least half the entire perimeter 15 of handle 14.

Referring to FIG. 2, installation of spray dispenser 10 begins by first positioning base 22 proximate to the door handle 14 (e.g., above the door handle on the door itself) and securing it to the door via screws 35. Next, component support wall 29 is mounted to base 22 using any conventional securing or locking means, such as screws, glue, Velcro, clips, snap-fit arrangements, etc. Finally, batteries 45 are inserted in battery compartment 44, germicide source 47 is inserted into mounting bracket 49 and outer cover 21 is fixedly attached to base 22 at support prongs 38. Outer cover 21 can now be closed providing complete cover for support wall 29 and the components thereon and spray dispenser 10 activated.

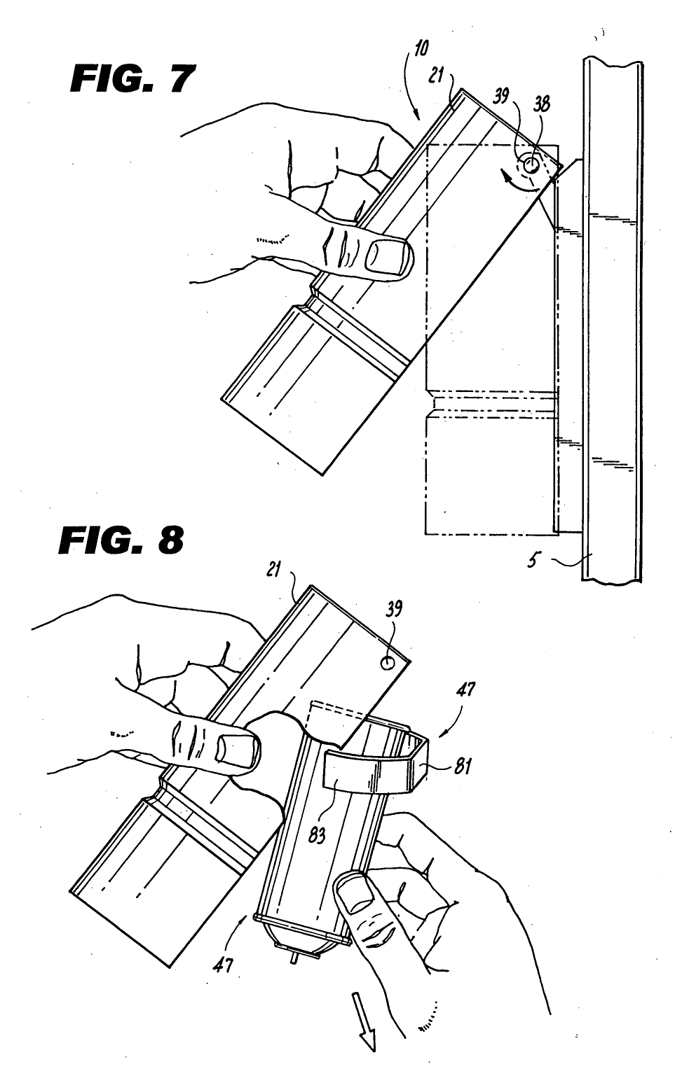

As further illustrated with reference to FIGS. 7 and 8, prongs 38 are configured to rest within orifices 39 of outer cover 21 and provide pivotal movement of the cover about the prongs 38, thereby permitting “opening” of spray dispenser 10 by pivoting cover 21 away from support 22 and “closing” of spray dispenser 10 by pivoting cover 21 toward support 22. Thus, complete access to both battery compartment 44 and germicide source 47 can be achieved. Optionally, the cover and support can be severed to one another with a lock or keyed release so as to prevent tampering by unauthorized individuals.

The germicide 2 contained in source 47 and sprayed on the handle 14 can comprise a disinfecting liquid, aerosol, mist or the like, and is preferably characterized by it's ability to sanitize surfaces it comes into contact with. According to alternative embodiments of the present of invention, germicide 2 comprises a chlorine-based chemical solution as this provides a relatively non-flammable fluid (as compared to an alcohol-based solution) and also provides the germicide function discussed above. It is to be understood that the term “germicide” used herein is not intended to be limited to any particular type of chemical solution or fluid and the scope and meaning of this term includes any suitable fluid or composition which acts as a disinfectant. Optionally, a scent additive may be included in the fluid in order that the sanitizing fluid may also function as an air freshener.

In certain instances, such as with room doors and the like, the spray dispenser 10 dispenses germicide 2 at certain given intervals in time, for example by a time switch, clock control, and the like. According to alterative aspects of the invention, the spray actuator 52 can be triggered in response to manipulation of the door handle 14, such as after each individual manipulation or following a given series of such manipulations. In the latter case, a counter can initiate the release of germicide 2 whenever a predetermined count is reached. Such cases just cited are all examples of a controlling software program. It is this software program that is responsible for automating the disinfecting methods disclosed earlier, by activating the handle sterilization process when a predetermined state or function is sensed.

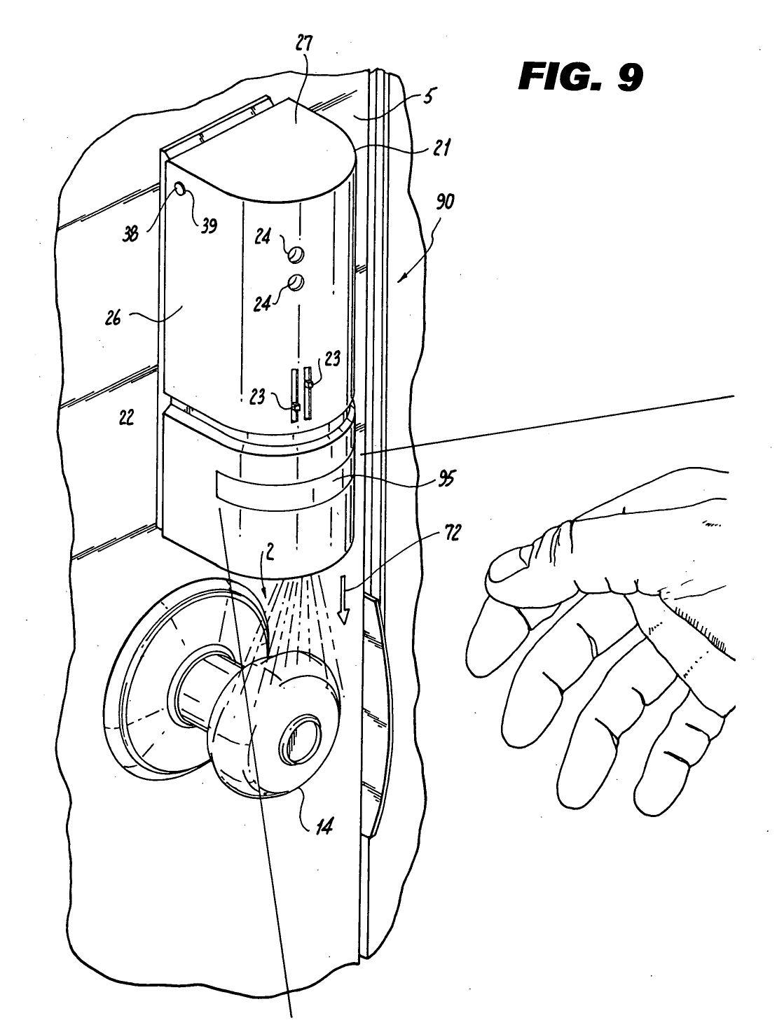

Referring to FIG. 9, according to a second embodiment of a door handle sanitizer, front wall 26 further comprises a sensor 95 for triggering spray actuator 52. Sensor 95 can be optical, infrared, mechanical/electrical or a combination of the above. Thus, in this arrangement the actuator 52 can activate the spray when a person or movement is detected in a vicinity of the handle or when a light beam interruption or vibration is detected.

Referring to FIG. 1, handle 14 can take any shape or size and is preferably configured to assist in opening and closing of door 5. Handle 14 can be of a stationary type used to push or pull door 5 or can be mechanically mounted and include a conventional locking mechanism, requiring rotation of a handle to unlock the door prior to opening and closing. While the handle is illustrated as a conventional cylindrical shaped door handle, the spray dispenser 10 described herein is operable to sanitize any door handle and examples of several handle shapes which can be sanitized by spray dispenser 10 include, but are not limited to, spherical, rectangular, hexagonal, rod or hook-shape handles and composites thereof. Handle 14 has a knob portion 13 comprising an outer perimeter 15 and a diameter dimension D which extends through the center of knob 13 to the outer perimeter 15. One of ordinary skill in the art would realize that door 5 can be handless, and in place of the handle a flat plate could be affixed to the surface of the door for pushing or pulling the door. As described in greater detail below, the embodiments of spray dispenser 10 described herein can be equally applied to such plate arrangements by orientating nozzle 56 to direct the germicide spray onto a plate-type arrangement as opposed to a protruding handle 14.

Although the embodiments of the invention were described with reference to a disinfection apparatus for restroom doors it should be noted that at least some of the embodiments are suitable also for disinfecting other kinds of handles, such as handles of cabinets for the sterile storing of surgical instruments, doors of operating theaters or rooms in hospitals etc.

While the invention has been described with reference to several embodiments thereof, the invention is more broadly defined and limited only by the recitations in the claims appended hereto and their legal equivalents.

I claim:

1. A door handle sanitizer for disinfecting a door handle comprising:
a source of liquid or gaseous disinfectant;
a nozzle fluidly connected to the source configured to convey liquid or gaseous disinfectant from the source to the nozzle and for dispensing the liquid or gaseous disinfectant onto the handle; and
a control circuit for causing the liquid or gaseous disinfectant to be conveyed to the handle in intervals, wherein the intervals are responsive to an external condition and the control circuit if free of any manual activation.

2. An apparatus proximate the handle of a door for dispensing germicide onto the handle and into the atmosphere surrounding the handle comprising:
a pressurized source of germicide having a normally closed, force actuated valve;
a valve actuating means for exerting a force on the valve sufficient to actuate the valve and release the germicide, the valve actuating means comprising:
a nozzle fluidly connected to the valve for transporting the germicide from the valve into the atmosphere surrounding the handle;
a mechanically driven arm or assembly connected to the valve, the nozzle, or both, operable to produce the force on the valve; and
a control circuit for activating the valve actuating means in intervals, wherein the intervals are responsive to an external condition.

3. A method for sanitizing a door handle of a door comprising the steps of:
mounting a dispenser unit proximate the door handle; and
spraying a germicide in an atomized mist from the dispenser onto the door handle in response to an event free of any manual activation.

4. The method of claim 3 further comprising the step of detecting the event.

5. The method of claim 4 further comprising the automated steps of:
supplying power to a motor in response to detecting the event; and
said motor effecting release of germicide onto the door handle when the motor is supplied with power.

6. The door handle sanitizer of claim 1 further comprising a means for mounting the sanitizer to the door.

7. The door handle sanitizer of claim 1 wherein the spray nozzle is configured to convey a prescribed amount of the liquid or gaseous disinfectant from the source to the nozzle and dispense the prescribed amount of liquid or gaseous disinfectant onto the handle.

8. The door handle sanitizer of claim 1 wherein the control circuit comprises a timing program which activates the nozzle to dispense the liquid or gaseous disinfectant at prescribed time intervals on the basis of a predetermined cleaning cycle of the door handle sanitizer.

9. The door handle sanitizer of claim 8 further comprising a manual switch connected to the control circuit, wherein the predetermined cleaning cycle is one of a plurality of cleaning cycles selectable by the switch.

10. The door handle sanitizer of claim 1 wherein the nozzle is configured to emit a fine mist in the atmosphere proximate the door handle.

11. The door handle sanitizer of claim 1 wherein the nozzle is orientatable toward a surface of the door handle for directing the liquid or gaseous disinfectant thereto.

12. The door handle sanitizer of claim 1 wherein the source of disinfectant is an aerosol germicide can and the nozzle dispenses a prescribed amount of aerosol germicide onto the door handle and into the atmosphere surrounding the handle.

13. The door handle sanitizer of claim 1 further comprising a sensor connected to the control circuit for sensing the external condition and providing an activation signal to the control circuit representative of the absence or occurrence of the external condition.

14. The door handle sanitizer of claim 1 further comprising a means for removably mounting the source of liquid or gaseous disinfectant within the sanitizer.

15. The apparatus of claim 2 wherein the pressurized source of germicide is an aerosol.

16. The apparatus of claim 2 wherein the force is one of a tilting, pushing or pulling force on the valve sufficient to actuate the valve.

17. The apparatus of claim 2 wherein the valve is an atomizing valve.

18. The door handle sanitizer of claim 13 wherein the sensor is a motion sensor with a prescribed range and the external condition is movement with the range.

19. The door handle sanitizer of claim 13 wherein the sensor is a infrared sensor and the external condition is any condition capable triggering the infrared sensor.

20. The apparatus of claim 2 further comprising a motor connected between the control circuit and the valve actuating means for driving the arm.
